(19) United States
(12) Patent Application Publication
(10) Pub. No.: US 2025/0160621 A1
(43) Pub. Date: May 22, 2025

(54) METHOD, PROGRAM, AND APPARATUS FOR GENERATING TORQUE FOR CONTROL OF ENDOSCOPY SCOPE

(71) Applicant: MedInTech Inc., Seoul (KR)

(72) Inventors: Chiwon LEE, Namyangju (KR); Myungjoon KIM, Gwacheon (KR); Geono KIM, Seoul (KR)

(21) Appl. No.: 18/953,251

(22) Filed: Nov. 20, 2024

(30) Foreign Application Priority Data
Nov. 20, 2023 (KR) 10-2023-0161497

(51) Int. Cl. A61B 1/00; A61B 1/005; G02B 23/24

(52) U.S. Cl. CPC A61B 1/00133; A61B 1/0051; G02B 23/2476

(57) ABSTRACT

According to one embodiment of the present, there is disclosed a method of generating torque for the control of an endoscopic scope that is performed by a computing device including at least one processor. The method includes: obtaining first data regarding the target movement state of an endoscopic scope included in an endoscope device and second data regarding the estimated movement state of the scope; and computing a torque for causing the scope to follow the position and speed desired by a user by using a mathematical model that uses the first data and the second data as input variables.

### CROSS-REFERENCE TO RELATED APPLICATION

This application claims the benefit of Korean Patent Application No. 10-2023-0161497 filed on Korean Intellectual Property Office, which is hereby incorporated by reference herein in its entirety.

### BACKGROUND

### 1. Technical Field

The present disclosure relates to endoscopic control technology, and more particularly, to a method, computer program, and apparatus for generating the torque required for controlling an endoscopic scope.

### 2. Description of the Related Art

Endoscopes collectively refer to medical devices that enable a scope to be inserted into the human body and a user to observe an organ without surgery or autopsy. Endoscopes enable a scope to be inserted into the body, radiate light, and visualize the light reflected from the surface of the inner wall of the body. Endoscopes are classified according to their purpose and target body part, and may be basically classified into rigid endoscopes, in which an endoscopic tube is made of metal, and flexible endoscopes, which are represented by digestive endoscopes.

A flexible endoscope contains various devices therein and is thus vulnerable to impact, and also the inside of a digestive organ into which a flexible endoscope is inserted corresponds to a considerably fragile tissue and has an irregular shape. Furthermore, the shape of the inside of the digestive organ varies depending on the patient, so that the process of inserting the endoscope may not be easy even for experienced medical professionals.

In this case, as an endoscopic procedure is performed, an endoscopic scope is inserted into a digestive organ while being twisted and turned according to the shape of the digestive organ. In the early stage of the endoscopic procedure, an endoscopic operator may bend or move the scope to a desired angle without a large amount of force because the scope has a relatively simple shape. However, as the procedure progresses to the latter half of the procedure or when complex movement is involved during the procedure, controlling the scope while taking into consideration the characteristics of the scope that may change at any moment can cause significant inconvenience to the endoscopic operator.

In addition, the nature of the endoscopic procedure in which the scope is inserted into the body requires the delicate control of the scope, and thus, there is a demand for technology for controlling an endoscopic scope by reflecting therein the physical changes that occur in an endoscope device during an endoscopic procedure.

### RELATED ART LITERATURE

### Patent Literature

Korean Patent No. 10-1940046 (published on Jan. 18, 2019)

### SUMMARY

The present disclosure is intended to overcome the problems of the above-described conventional art, and is directed to a method, a computer program, and an apparatus for compensating the torque generated in an endoscope device so that the scope can move to a position desired by a user at a speed also desired by the user.

However, objects to be achieved by the present disclosure are not limited to the object described above, and another object may be present.

According to one embodiment of the present disclosure for achieving the above-described object, there is disclosed a method of generating torque for the control of an endoscopic scope that is performed by a computing device including at least one processor. The method includes: obtaining first data regarding the target movement state of an endoscopic scope included in an endoscope device and second data regarding the estimated movement state of the scope; and the computing a torque for causing the scope to follow position and speed desired by a user by using a mathematical model that uses the first data and the second data as input variables.

Alternatively, the target movement state of the scope may include at least one of the target position, target speed, and target acceleration of the scope, which are calculated through a command using a manipulation portion included in the endoscope device.

Alternatively, the estimated movement state of the scope may include at least one of the estimated position, estimated velocity, and estimated acceleration of the scope, which are calculated based on the actual position data of a motor included in the endoscope device and measured by a sensor, the actual velocity data of the motor measured by the sensor, and the target velocity data of the scope included in the first data.

Alternatively, the mathematical model may include a combination of dynamic terms based on sliding mode control.

Alternatively, the dynamic terms may include: a first term regarding inertia using the estimated position of the scope included in the second data as an input variable; a second term regarding Coriolis force using the estimated position of the scope and the estimated speed of the scope included in the second data as input variables; and a third term regarding force attributable to the shape of the scope using the estimated position of the scope as an input variable.

Alternatively, the combination of the dynamic terms may be derived by combining a basic equation including the sum of the first, second, and third terms and a Lyapunov function having a semi-definite form.

Alternatively, the Lyapunov function may include a detailed term for reflecting therein a spring effect generated by a string of the scope.

Alternatively, the Lyapunov function may be made into a semi-definite form by reflecting therein an equation for a sliding surface that uses the difference between the second data and the first data as an input variable.

Alternatively, the Lyapunov function may be made into a semi-definite code form by reflecting therein an equation based on a sign function for compensating for the maximum value of uncertainty attributable to disturbance and an equation for reducing tracking errors that are likely to occur in the process of compensating for the maximum value.

According to one embodiment of the present disclosure for achieving the above-described object, there is disclosed a computer program stored in a computer-readable storage medium, the computer program performing operations for controlling an endoscopic scope when executed on one or more processors. The operations include operations of: obtaining first data regarding the target movement state of an endoscopic scope included in an endoscope device and second data regarding the estimated movement state of the scope; and computing a torque for causing the scope to follow the position and speed desired by a user by using a mathematical model that uses the first data and the second data as input variables.

According to one embodiment of the present disclosure for achieving the above-described object, there is disclosed a computing device for generating torque for the control of an endoscopic scope. The computing device includes: a processor including at least one core; and memory including a force model, in which at least one parameter has been identified, and program codes executable on the processor. The processor obtains first data regarding the target movement state of an endoscopic scope included in an endoscope device and second data regarding the estimated movement state of the scope, and computes a torque for causing the scope to follow the position and speed desired by a user by using a mathematical model that uses the first data and the second data as input variables.

According to one embodiment of the present disclosure, the torque that causes the scope of the endoscope device to follow the position and speed desired by a user is generated, so that precise and fine scope control can be performed. That is, the error between the manipulation of a user and the movement of the scope is reduced, so that the convenience and satisfaction of an endoscopic procedure can be increased and a sense of stability can be provided to a patient during an endoscopic procedure.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a diagram showing the configuration of endoscope device according to one embodiment of the present disclosure;

FIG. 2 is a block diagram showing a computing device according to one embodiment of the present disclosure;

FIG. 3 is a block diagram showing a torque computation process according to one embodiment of the present disclosure; and

FIG. 4 is a flowchart illustrating a torque generation method for controlling an endoscope device according to one embodiment of the present disclosure.

### DETAILED DESCRIPTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, a singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit that processes computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The term “acquiring” used herein may be understood to mean not only receiving data over a wired/wireless communication network with an external device or system, but also generating data in an on-device form.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

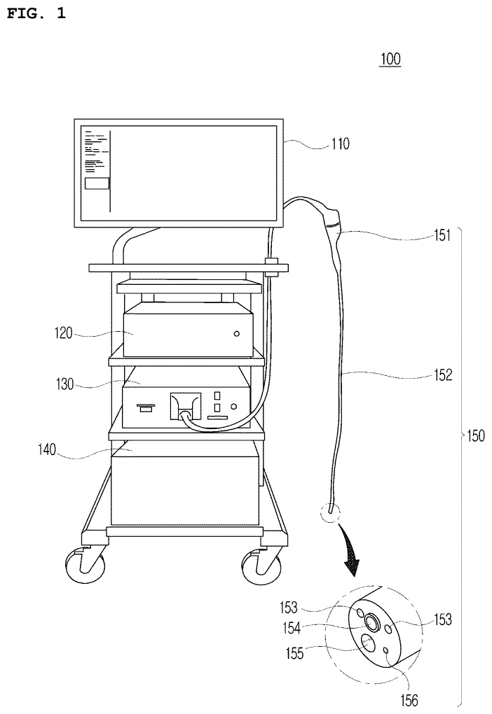

FIG. 1 is a diagram showing the configuration of endoscope device according to one embodiment of the present disclosure.

Referring to FIG. 1, an endoscope device 100 according to one embodiment of the present disclosure may be a flexible endoscope, or more specifically, a digestive endoscope. The endoscope device 100 may include a configuration capable of obtaining a medical image adapted to photograph the inside of a digestive organ and a configuration capable of, when necessary, allowing a tool to be inserted and a user to perform treatment or manipulation while viewing a medical image.

The endoscope device 100 may include an output unit 110, a control unit 120, a drive unit 130, a pump unit 140, and a scope 150, and may further include a light source unit (not shown).

The output unit 110 may include a display configured to display medical images. The output unit 110 may include a display module configured to output visualized information or implement touch screens, such as a liquid crystal display (LCD), a thin film transistor-liquid crystal display (TFT LCD), an organic light-emitting diode (OLED), a flexible display, a three-dimensional (3D) display, or the like.

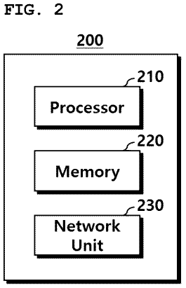

The output unit 110 may include various means for providing medical images or information about medical images. The output unit 110 may display medical images obtained by the scope 150 or medical images processed by the control unit 120. The output unit 110 may provide information through an auditory means in addition to a visual means, and may include, for example, a speaker configured to provide alarms regarding medical images audibly. Meanwhile, although the one output unit 110 is illustrated in FIG. 2, a plurality of output units 110 may be provided. In this case, an output unit 110 where a medical image obtained by the scope 150 is displayed and an output unit 110 where information processed by the control unit 120 is displayed may be separated from each other.

The control unit 120 may control the overall operation of the endoscope device 100. For example, the control unit 120 may perform the operation of taking a medical image through the scope 150, the operation of processing an obtained medical image, the control operation of performing medical operations such as the spraying of washing water, suction, or the like, a series of operations for controlling the movement of the scope 150, etc. The control unit 120 may include all types of devices capable of processing data. According to an exemplary embodiment, the control unit 120 may be a data processing device embedded in hardware that has circuits physically structured to perform the functions represented by the codes or instructions included in a program. As an example of the data processing device embedded in hardware, a processing device such as a microprocessor, a central processing unit (CPU), a processor core, a multiprocessor, an application-specific integrated circuit (ASIC), or a field programmable gate array (FPGA) may be included, but the technical spirit of the present disclosure is not limited thereto.

The control unit 120 may control the movement of the scope 150 through the drive unit 130 connected to the scope 150. That is, the control unit 120 may generate control signals to be provided to the drive unit 130 in order to control the movement of the scope 150.

For example, a series of operations in which the endoscope device 100 of the present disclosure controls the scope 150 may be performed as follows. A user may input the degree or direction of curvature of the scope 150 through a manipulation portion. The input information is transmitted to the control unit 120, and the control unit 120 may process the input information and generate a signal to be provided to the drive unit 130. For example, the control unit 120 may calculate the position, angle, angular velocity, and/or like of a motor corresponding to the degree or direction of curvature set by the user and may provide them to the drive unit 130. The drive unit 130 may generate power based on the signal of the control unit 120 and transmit it to the scope 150. Accordingly, the scope 150 may be moved or bent in accordance with the value input by the user.

The drive unit 130 may provide power required for the scope 150 to be inserted into the body, bent, and moved inside the body. For example, the drive unit 130 may include the motor connected to the wire inside the scope 150 and a tension adjustment unit configured to adjust the tension of the wire.

The drive unit 130 may control the scope 150 in various directions by controlling the power of the motor. For example, the motor may be configured to include a plurality of motors corresponding to the directions in which the insertion portion at the end of the scope 150 is to be bent. Alternatively, the motor may be configured to include a plurality of motors corresponding to wires inside the scope 150. More specifically, the drive unit 130 may include a first motor configured to determine the x-axis movement of the scope 150 and a second motor configured to determine the y-axis movement of the scope 150. The x-axis position, y-axis position, roll, pitch, and yaw values of the end of the scope 150 may be determined according to the control of the drive unit 130, but the configuration of the drive unit 130 is not limited thereto.

The tension adjustment unit may receive power from the motor and pull the wire inside the scope 150 to generate tension. Through this, the scope 150 may be bent. The tension adjustment unit 330 may adjust the tension applied to the plurality of wires 1000 inside the scope 150 so that the scope 150 can be bent according to the determined amount and direction of bending.

The pump unit 140 may include at least one of an air pump configured to inject air into the human body through the scope 150, a suction pump configured to provide negative pressure or vacuum and suck air from the body through the scope 150, and a water pump configured to inject cleaning water into the body through the scope 150. Each of the pumps may include a valve configured to control the flow of fluid. The pump unit 140 may be opened and closed by the control unit 120. At least one of the suction pump, the water pump, and the air pump may be opened and closed based on a control signal of a computing device or the control of the control unit 120.

The scope 150 may include the insertion portion 152 configured to be inserted into a digestive organ and the operating portion 151 configured to control the movement of the insertion portion 152 and receive input from a user to perform various operations.

The insertion portion 152 is configured to be flexibly bent and is connected to the drive unit 130 at one end thereof, so that the degree or direction of curvature can be determined by the drive unit 130. Since medical imaging and treatment are performed at the end of the insertion portion 152, the scope 150 may include various cables and tubes that extend to the end of the insertion portion 152. A light source lens 153, an objective lens 154, a working channel 155, and an air and water channel 156 may be provided inside the scope 150. A tool for treating and managing a lesion may be inserted through the working channel 155 during an endoscopic procedure. Air may be injected and washing water may be fed through the air and water channel 156. Meanwhile, in FIG. 2, the air and water channel 156 is illustrated as a passage through which washing water is fed, but it is not limited thereto. For example, a separate water jet channel (not shown) may be provided inside the scope 150, and cleaning water may also be fed through a water jet channel.

Meanwhile, references herein to the scope 150 being bent by the control unit 120 or the drive unit 130 may refer to at least a part of the scope 150, e.g., the insertion portion 152, being bent.

The manipulation portion 151 may include a plurality of input buttons configure to provide various functions (image capture, the spray of cleaning water, etc.) to allow an endoscopic operator to control the steering of the insertion portion 152 and perform a procedure through the working channel 155 and the air and water channel 156. For example, the manipulation portion 151 may include a plurality of buttons or a joystick-type input device configured to indicate the direction of the scope 150.

The light source unit may include a light source that radiates light into the body through the endoscopic scope 150. The light source unit may include a lighting device configured to generate white light, or may include a plurality of lighting devices configured to generate rays of light having different wavelength bands. The type of light source, the intensity of light, white balance, and/or the like may be set through the light source unit. Meanwhile, the above-described setting items may also be set through the control unit 120. The light generated by the light source unit may be transmitted to the scope 150 through a path such as an optical fiber.

FIG. 2 is a block diagram showing a computing device according to one embodiment of the present disclosure.

The computing device 200 according to the one embodiment of the present disclosure may be a hardware device or part of a hardware device that performs the comprehensive processing and computation of data, or may be a software-based computing environment that is connected to a communication network. For example, the computing device 200 may be a dependent device that is built into an endoscope device and performs an intensive data processing function required for controlling the endoscope device. The computing device 200 may also be an independent device, such as a server, that performs an intensive data processing function required for controlling an endoscope device via wired/wireless communication with the endoscope device. Since the foregoing description is only one example related to the type of computing device 200, the type of computing device 200 may be configured in various manners within a range understandable to those skilled in the art based on the content of the present disclosure.

Referring to FIG. 2, the computing device 200 according to the one embodiment of the present disclosure may include a processor 210, memory 220, and a network unit 230. However, FIG. 2 illustrates only an example, and the computing device 200 may include other components for implementing a computing environment. Furthermore, only some of the components disclosed above may be included in the computing device 200.

The processor 210 according to an embodiment of the present disclosure may be understood as a constituent unit including hardware and/or software for performing computing operation. For example, the processor 210 may read a computer program and perform the processing of data obtainable in the process of controlling an endoscope device. The processor 210 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 210 described above are only examples, the type of processor 210 may be configured in various manners within a range understandable to those skilled in the art based on the content of the present disclosure.

The processor 210 may compute the torque that causes the scope of the endoscope device to follow the position and speed desired by a user. For example, when the end of the scope 150 and the drive unit 130 are separated by a specific distance or more as in the endoscope device 100 of FIG. 1, the power of the drive unit 130 may not be completely transmitted to the end of the scope 150 through the wire, and thus, an error may occur between the user's control command and the movement of the scope 150. The processor 210 may compute the torque required to reduce the error between the control command and the movement of the scope 150 by performing computation based on data regarding the target movement state of the scope 150 and data regarding the estimated movement state of the scope 150. In this case, the computation performed by the processor 210 may be performed using a mathematical model that computes a torque value by reflecting the physical characteristics of the endoscope device 100 therein.

The memory 220 according to an embodiment of the present disclosure may be understood as a constituent unit including hardware and/or software for storing and managing data that is processed in the computing device 200. That is, the memory 220 may store any type of data generated or determined by the processor 210 and any type of data received by the network unit 230. For example, the memory 220 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 220 may include a database system that controls and manages data in a predetermined system. Since the types of memory 220 described above are only examples, the type of memory 220 may be configured in various manners within a range understandable to those skilled in the art based on the content of the present disclosure.

The memory 220 may structure, organize, and manage data required for the processor 110 to perform computation, the combinations of data, and program codes executable on the processor 210. For example, the memory 220 may store program codes adapted to operate the processor 210 to compute torque using a mathematical model, and may also store various types of data generated as the program codes are executed. Furthermore, the memory 220 may store various types of data transmitted and received via the network unit 230 to be described below for use in the torque computation of the processor 210.

The network unit 230 according to an embodiment of the present disclosure may be understood as a constituent unit that transmits and receives data through any type of known wired/wireless communication system. For example, the network unit 230 may perform data transmission and reception using a communication system such as a local area wired/wireless network (LAN), a wideband code division multiple access (WCDMA) network, a long term evolution (LTE) network, the wireless broadband Internet (WiBro), a 5th generation mobile communication (5G) network, a ultra wide-band wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 230 may be applied in various manners other than the above-described examples.

Meanwhile, data to be processed by the processor 210 may be stored in the memory 220 or received via the network unit 230, and data generated by the processor 210 may be stored in the memory 220 or transmitted to the outside via the network unit 230.

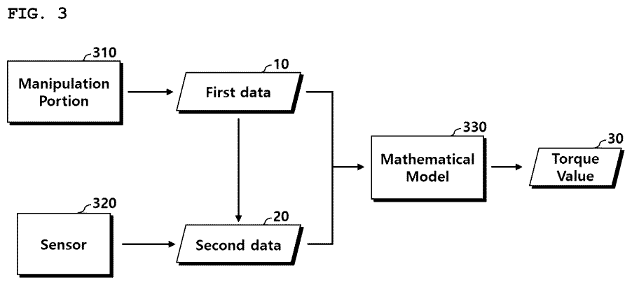

FIG. 3 is a block diagram showing a torque computation process according to one embodiment of the present disclosure.

Referring to FIG. 3, a computing device according to one embodiment of the present disclosure may obtain first data 10 regarding the target movement state of a scope included in an endoscope device. In this case, the first data 10 may be generated through a command using a manipulation portion 310 included in the endoscope device. When a user inputs a command using the manipulation portion 310, the computing device may obtain the first data 10 according to the command input through the manipulation portion 310. In this case, the first data 10 may include at least one of data regarding the target position of the scope, data regarding the target speed of the scope, and data regarding the target acceleration of the scope.

The computing device may obtain second data 20 regarding the estimated movement state of the scope. In this case, the second data 20 may be generated from the results detected by a sensor 320 that detects the movement of a motor included in the endoscope device. It is difficult to directly equip the scope with a sensor to estimate the movement state of the scope due to the nature and structure of the scope that is inserted into the human body. Since the movement of the scope is affected by the movement of the motor, the computing device may use the actual position data of the motor measured by the sensor 320 and the actual speed data of the motor measured by the sensor 320 to obtain the second data 20. For example, the computing device may compute the second data 20 based on the actual position data of the motor, the actual speed data of the motor, and the target speed data of the scope included in the first data 10. The computing device may compute the second data 20 by inputting the actual position data of the motor, the actual speed data of the motor, and the target speed data of the scope included in the first data 10 to a pre-trained machine learning model. In this case, the second data 20 may include at least one of data regarding the estimated position of the scope, data regarding the estimated speed of the scope, and data regarding the estimated acceleration of the scope. The machine learning model may be a model that is trained through supervised learning, self-supervised learning, or unsupervised learning. Alternatively, the machine learning model may include a plurality of neural networks that are structured according to respective learning methodologies. Meanwhile, since the estimation using the machine learning model is only one example regarding the estimation of the movement state of the scope, the present disclosure is not limited to this example.

The computing device may generate a torque value 30 required for the scope to follow the position and speed desired by the user by using a mathematical model 330 that uses the first data 10 and the second data 20 as input variables. In this case, the mathematical model 330 may be composed of a combination of dynamic terms based on sliding mode control. The dynamic terms may include a first term regarding inertia using the estimated position of the scope included in the second data 20 as an input variable, a second term regarding Coriolis force using the estimated position of the scope and the estimated speed of the scope included in the second data as input variables, and a third term regarding force attributable to the shape of the scope using the estimated position of the scope an as input variable. Furthermore, the combination of dynamic terms may be derived by combining a basic equation including the sum of the first, second and third terms, and a Lyapunov function having a semi-definite form.

More specifically, the basic equation for the combination of dynamic terms constituting the mathematical model 300 may be represented by Equation 1 below:

$$M ( q ) \ddot{q} + C ( q , \dot{q} ) \dot{q} + K ( q ) = τ + Δ f \qquad ( 1 )$$

In this case, M(q)\ddot{q} denotes the first term including term M regarding inertia, C(q,\dot{q})\dot{q} denotes the second term including term C regarding the Coriolis force, and K(q) denotes the third term including term K regarding the force attributable to the shape of the scope. Furthermore, q denotes the estimated position of the scope, \dot{q} denotes the estimated velocity of the scope, and \ddot{q} denotes the estimated acceleration of the scope. Moreover, τ denotes the torque, and Δf denotes uncertainty attributable to disturbance.

The basic form of the Lyapunov function for the combination of dynamic terms constituting the mathematical model 300 may be represented by Equation 2 below:

$$V = \frac{1}{2} {s}^{T} Ms + \frac{1}{2} {s}^{T} Ks \qquad ( 2 )$$

In this case, s denotes the sliding surface. Referring to Equation 2, the basic form of the Lyapunov function includes a detailed term ½s^{T}Ks adapted to reflect the spring effect caused by the string (or the wire) of the scope. This corresponds to a unique concept derived because it is necessary to reflect the spring effect, generated in a control process as the string connecting the driving unit included in the endoscope device and the end of the scope becomes longer, in the computation of the torque.

The basic form of the Lyapunov function described above may be made into a semi-definite form by reflecting therein an equation for a sliding surface that uses the difference between the second data 20 and the first data 10 as an input variable, an equation based on a sign function for compensating for the maximum value of uncertainty attributable to disturbance, and an equation for reducing tracking errors that may occur in the process of compensating for the maximum value.

The equation for a sliding surface that uses the difference between the second data 20 and the first data 10 as an input variable may be represented by Equation 3 below:

$$\dot{s} = \ddot{e} + λ \dot{e} = \ddot{q} - \ddot{{q}_{d}} + λ ( \dot{q} - \dot{{q}_{d}} ) \qquad ( 3 )$$

In Equation 3, \dot{q}_{d} denotes the target velocity of the scope, and \ddot{q}_{d} denotes the target acceleration of the scope. In order to make the Lyapunov function into a semi-definite form, when Equation 3 is substituted into Equation 2 and then rearranged, Equation 4 below is obtained:

$$\dot{V} = {s}^{T} ( ( M + K ) ( {M}^{- 1} τ + {M}^{- 1} Δ f - {M}^{- 1} C \dot{q} - {M}^{- 1} K - \ddot{{q}_{d}} + λ ( \dot{q} - \dot{{q}_{d}} ) ) + ( C + \frac{1}{2} \dot{K} ) s ) \qquad ( 4 )$$

In addition, in order to design the input so that the Lyapunov function can be made into a semi-definite form, when τ that eliminates the terms for M, C, and G excluding Δf in Equation 4 is constructed, it may be derived as in Equation 5 below:

$$τ = M ( \ddot{{q}_{d}} - λ ( \dot{q} - \dot{{q}_{d}} ) ) - {M ( M + K )}^{- 1} ( C + \frac{1}{2} \dot{K} ) ( ( \dot{q} - \dot{{q}_{d}} ) + λ ( q - {q}_{d} ) ) + C \dot{q} + K - Δ f \qquad ( 5 )$$

In this case, in order to additionally design the input so that the Lyapunov function can finally be made into a semi-definite sign, Equation 6, which is an equation based on a sign function for compensating for the maximum value of uncertainty attributable to disturbance and an equation for reducing the tracking errors that may occur in the process of compensating for the maximum value, may be reflected in Equation 5.

$$α = - ρ sign ( s ) - η s \qquad ( 6 )$$

In this equation, −ρsign(s) denotes a formula for compensating for the maximum value of uncertainty. −ηs denotes a formula for reducing the tracking errors that may occur because the differential value of the Lyapunov function may become 0 when the maximum disturbance is compensated for.

The results of reflecting additional input α in Equation 5 may be represented by Equation 7 below:

$$τ = M ( \ddot{{q}_{d}} - λ ( \dot{q} - \dot{{q}_{d}} ) ) - {M ( M + K )}^{- 1} ( C + \frac{1}{2} \dot{K} ) ( ( \dot{q} - {q}_{d} ) + λ ( q - {q}_{d} ) ) + C \dot{q} + K - ρ sign ( s ) - η s \qquad ( 7 )$$

Equation 7 may correspond to a combination of dynamic terms that constitute the mathematical model 300. The computing device may compute a torque capable of minimizing the end tracking errors that may occur during the process of controlling the scope by using the mathematical model 300 reflecting the physical characteristics of the endoscope device therein.

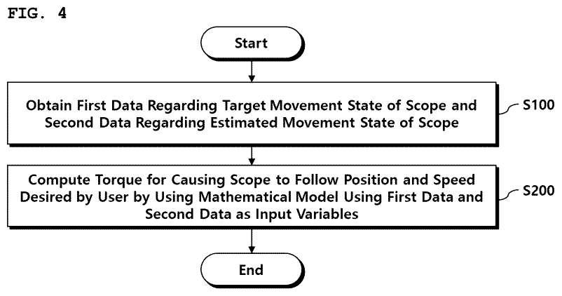

FIG. 4 is a flowchart illustrating a torque generation method for controlling an endoscope device according to one embodiment of the present disclosure.

Referring to FIG. 4, a computing device according to one embodiment of the present disclosure may obtain first data regarding the target movement state of the scope included in the endoscope device and second data regarding the estimated movement state of the scope in step S100. For example, when the computing device is the control unit included in the endoscope device, the computing device may generate first data and second data by processing the data obtained through the manipulation portion and sensor included in the endoscope device to. When the computing device is an external device independent of the endoscope device, the computing device may receive the data obtained through the manipulation portion and the sensor via wired/wireless communication with the endoscope device. Furthermore, the computing device may generate first data and second data by processing the received data.

The computing device may compute the torque required for the scope to follow the position and speed desired by a user by using a mathematical model that uses the first data and the second data as input variables in step S200. The computation process for computing a torque is specifically described in conjunction with FIG. 3, so that a description thereof is omitted below.

The description of the present disclosure described above is intended for illustrative purposes, and those of ordinary skill in the art to which the present disclosure pertains can appreciate that the present disclosure may be easily modified into other specific forms without changing the technical spirit or essential features of the present disclosure. Therefore, it should be understood that the embodiments described above are illustrative and not restrictive in all respects. For example, each component described as being in a single form may be implemented in a distributed form. In the same manner, components described as being in a distributed form may be implemented in a combined form.

What is claimed is:

1. A method of generating torque for control of an endoscopic scope, the method being performed by a computing device including at least one processor, the method comprising:
obtaining first data regarding a target movement state of an endoscopic scope included in an endoscope device and second data regarding an estimated movement state of the scope; and
computing a torque for causing the scope to follow a position and speed desired by a user by using a mathematical model that uses the first data and the second data as input variables,
wherein the mathematical model includes a combination of dynamic terms based on sliding mode control, and
wherein the dynamic terms include a third term regarding force attributable to a shape of the scope using estimated position of the scope included in the second data as an input variable.

2. The method of claim 1, wherein the target movement state of the scope includes at least one of a target position, target speed, and target acceleration of the scope, which are calculated through a command using a manipulation portion included in the endoscope device.

3. The method of claim 1, wherein the estimated movement state of the scope includes at least one of the estimated position, estimated velocity, and estimated acceleration of the scope, which are calculated based on actual position data of a motor included in the endoscope device and measured by a sensor, actual velocity data of the motor measured by the sensor, and target velocity data of the scope included in the first data.

4. The method of claim 1, wherein the dynamic terms more include:
a first term regarding inertia using the estimated position of the scope included in the second data as an input variable; and
a second term regarding Coriolis force using the estimated position of the scope and an estimated speed of the scope included in the second data as input variables.

5. The method of claim 4, wherein the combination of the dynamic terms is derived by combining a basic equation including a sum of the first, second, and third terms and a Lyapunov function having a semi-definite form.

6. The method of claim 5, wherein the Lyapunov function includes a detailed term for reflecting therein a spring effect generated by a string of the scope.

7. The method of claim 5, wherein the Lyapunov function is made into a semi-definite form by reflecting therein an equation for a sliding surface that uses a difference between the second data and the first data as an input variable.

8. The method of claim 7, wherein the Lyapunov function is made into a semi-definite code form by reflecting therein an equation based on a sign function for compensating for a maximum value of uncertainty attributable to disturbance and an equation for reducing tracking errors that are likely to occur in a process of compensating for the maximum value.

9. A computer program stored in a computer-readable storage medium, the computer program performing operations for controlling an endoscopic scope when executed on one or more processors,
wherein the operations include operations of:
obtaining first data regarding a target movement state of an endoscopic scope included in an endoscope device and second data regarding an estimated movement state of the scope; and
computing a torque for causing the scope to follow a position and speed desired by a user by using a mathematical model that uses the first data and the second data as input variables,
wherein the mathematical model includes a combination of dynamic terms based on sliding mode control, and
wherein the dynamic terms include a third term regarding force attributable to a shape of the scope using estimated position of the scope included in the second data as an input variable.

10. A computing device for generating torque for control of an endoscopic scope, the computing device comprising:
a processor including at least one core; and
memory including a force model, in which at least one parameter has been identified, and program codes executable on the processor;
wherein the processor obtains first data regarding a target movement state of an endoscopic scope included in an endoscope device and second data regarding an estimated movement state of the scope, and computes a torque for causing the scope to follow a position and speed desired by a user by using a mathematical model that uses the first data and the second data as input variables,
wherein the mathematical model includes a combination of dynamic terms based on sliding mode control, and
wherein the dynamic terms include a third term regarding force attributable to a shape of the scope using estimated position of the scope included in the second data as an input variable.
